(19) United States
(12) Patent Application Publication
(10) Pub. No.: US 2025/0288492 A1
(43) Pub. Date: Sep. 18, 2025

(54) VIAL ADAPTER AND INJECTOR ASSEMBLY FOR A NEEDLE-FREE INJECTOR

(71) Applicant: Medical International Technologies (MIT Canada) Inc., Saint-Laurent, Québec (CA)

(72) Inventors: Karim Menassa, Saint-Laurent, Québec (CA); Maurice Menassa, Pierrefonds, Québec (CA); Adam Gadoua, Laval, Québec (CA)

(73) Assignee: Medical International Technologies (MIT Canada) Inc., Saint-Laurent, Québec (CA)

(21) Appl. No.: 18/861,326

(22) PCT Filed: Apr. 27, 2023
(86) PCT No.: PCT/CA2023/050567

§ 371 Date: Oct. 29, 2024

Related U.S. Application Data
(60) Provisional application No. 63/336,437, filed on Apr. 29, 2022.

(51) Int. Cl. A61J 1/20; A61J 1/06; A61J 1/14; A61M 5/30; A61M 19/00

(52) U.S. Cl. CPC A61J 1/2048; A61J 1/065; A61J 1/1406; A61J 1/201; A61M 5/30; A61M 19/00; A61M 2210/0631

(57) ABSTRACT

A needle-free injector assembly and vial adapter therefor is provided. The assembly comprises barrel containing a shaft assembly, a power injection unit attached at one end of the barrel, and a vial adapter for holding a vial of medicament to be injected connected at another end. The vial adapter comprising a vial cartridge holder with a protector tube for holding the vial of medicament, a cutting means for perforating the vial seal, and a locking connection with the assembly nozzle. The assembly may be used in dental applications, such as for the administration of anesthetic to oral tissue.

### CROSS-REFERENCE TO RELATED APPLICATIONS

The present application claims priority to U.S. Provisional Patent Application Ser. No. 63/336,437 filed Apr. 29, 2022, the entire contents of which are incorporated herein by reference.

### FIELD

The present application generally relates to needleless (needle-free) injectors and accessories therefor. More particularly, the present application relates to a vial adapter and assembly for a needle-free injector.

### BACKGROUND

Needle-free (or needleless) injectors and accessories thereof are known in the art (see, for example, U.S. Pat. No. 7,357,915 to Menassa; U.S. Pat. No. 9,662,460 to Menassa; U.S. Pat. No. 9,498,407 to Menassa; U.S. Pat. No. 9,067,020 to Menassa; U.S. Pat. No. 9,067,019 to Menassa; U.S. Pat. No. 4,623,332 to Lindmayer; U.S. Pat. No. 4,342,310 to Lindmayer). Such injectors typically include a chamber within a barrel for receiving or storing a liquid medicament at a proximal (injecting) end, and a plunger and piston combination positioned at a rear (distal) end of the barrel which acts under pressure by compressed gas behind the piston. On actuation of a trigger or button, the gas is released with sufficient force to act against the piston and expel the medicament, out of the barrel through an orifice at a tip of the opposite proximal end of the chamber, and through the skin of a user (human or other animal) intending to receive the medicament. Injectors can be single-use (i.e., a single medicament for administration to a single user), but others may be reusable once the medicament has been dispensed.

In certain injectors, a cartridge containing the liquid medicament is added to the chamber. Many cartridges are typically disposable after a single use. For health and safety reasons, as well as convenience, disposable single-use cartridges are ideal to reduce cross-contamination between different users.

Needle-free injectors are used in many different health applications. In dental applications, for example, liquid medicament (such as an anesthetic) is typically injected into oral tissue in a patient using an injection device comprising a modified syringe and needle attached at one end. A typical gun-type syringe and needle is shown in U.S. Pat. No. 3,905,365 to Colombo. U.S. Pat. No. 5,437,647 to Firth et al. shows a syringe and a protective case for holding a carpule containing a medicament for injecting into a patient through a needle. Other needle-free injectors are known as in U.S. Pat. No. 5,501,666 to Spielberg and EP 0 294 272 A2 to Accaries. In some examples, a dental syringe has a compartment for holding a glass vial of medicament, with a plunger which is connected at one end with a modified hand/finger adapter to facilitate pushing on plunger to expel the medicament, and a needle at the other end of the compartment (see FIG. 1). Devices of the like used in dental applications have their limitations.

For many patients, dental needles can be quite unpleasant, painful and/or stressful. Many patients may avoid or are otherwise dread dental visits, particularly when faced with the prospect of having to receive a needle during a dental procedure, thereby failing to give proper regular attention to their oral and dental health needs. This fear and neglect can lead to further health complications.

Many current injection devices, particularly in dental applications, require delicate, skilled handling by a trained professional to ensure an effective delivery of medicament to the tissues in the patient's oral cavity. Usually, glass vials containing the medicament are used. As such, there is an increased chance of vial breakage which can put the safety of the patient and dentist at risk.

There is a need for an injection device for administering a medicament, particularly in dental applications, that is potentially safer to use, less painful, less unpleasant and less intimidating to patients. In particular, there is a need to provide a device that reduces the risk of vial breakage and cross-contamination, especially when used in the mouth of a patient.

This background information is provided for making information available believed by the applicant to be of possible relevance to the present application. No admission is necessarily intended, nor should be construed, that any of the preceding information constitutes prior art against the subject matter presented herein.

### SUMMARY

An aspect of the present application is to provide a vial adapter and assembly for a needle-free injector.

In accordance with one aspect, there is provided a needle-free injector assembly comprising: a barrel attached at a first end to a power injector unit having an impactor shaft therein; a vial adapter releasably connected at or insertable into a second end of the barrel for housing a vial (such as a glass vial) of medicament to be injected inserted preferably at a rear end thereof; and an injection nozzle for releasably attaching to a front injection end of the vial adapter.

In another aspect, there is provided a needle-free injector assembly comprising: a barrel, such as a single machined barrel, releasably connected to a power injection unit, the barrel comprising a barrel shaft for housing an impactor shaft from a power injection unit at the rear end, and a vial adapter component at an injection end (such as, in one example, at the front end) of the barrel for housing a vial adapter comprising a vial or carpule of medicament; and an injection nozzle which is releasably attachable to the injection end of the barrel, whereby when attaching the nozzle to the injection end, a spike perforates a seal on the vial thereby releasing medicament.

In another aspect, there is provided a vial adapter for a needle-free injector comprising: a vial cartridge holder comprising a protector tube (typically made of metal or durable plastic, etc., which is less likely to break in normal usage) for holding a vial of medicament to be injected; a cutting means, such as a spike, knife or other sharp, for perforating a seal on the vial to form a perforation which allows the medicament to be injected therethrough; and a luer lock at one end of the adapter for releasably connecting with an injection nozzle.

In some embodiments, the vial adapter and assembly described herein are used for dental applications, such as a dental tool for injecting medicament (e.g. anesthetic) into the oral tissue of a patient. The vial adapter can be a single unit comprising an adapter portion which is connected to the injector impactor shaft and a compartment (barrel) for holding a vial adapter body and vial, or it may be a combination of separate injector adapter portion and barrel for holding the vial adapter body and vial.

In some embodiments, there is provided a dental tool releasably attachable to a needle-free injector power unit comprising a barrel for housing a piston/rod assembly which is impacted by the power unit, and for housing medicament which is aspirated/siphoned therein, for injection through a nozzle end of the tool.

### BRIEF DESCRIPTION OF THE DRAWINGS

FIG. 1 illustrates a typical dental syringe and needle.

FIG. 2 illustrates the dental syringe of FIG. 1 without the needle and nozzle.

FIG. 3 illustrates a vial (cartridge) adapter for use with a needle free injector as described herein.

FIG. 4 illustrates a vial adapter from FIG. 3 with the dust cap and plunger adapter removed.

FIG. 5 illustrates a selection of dental injection devices.

FIG. 6 illustrates a tip for use with an injector adapter device.

FIG. 7 illustrates one example of dental needle-free stainless steel cartridge and piston for administering medicament with a needle-free injector.

FIG. 8 illustrates one example of a dental needle-free stainless steel cartridge and piston for administering medicament with a needle-free injector.

FIG. 9 illustrates one example of dental needle-free stainless steel cartridge and piston for administering medicament with a needle-free injector.

FIG. 10 illustrates a dental needle-free plastic disposable cartridge for holding a medicament for use with a needle-free injector.

FIG. 11 illustrates a vial cartridge for holding an ampoule of medicament for use with a needle-free injector.

FIGS. 12-14 illustrate embodiments of a vial adapter (e.g., glass vial adapter) that may be used in with a needle-free injector.

FIG. 15 illustrates a plunger cap for a vial adapter as described herein.

FIG. 16 illustrates an exploded view of a vial adapter with glass medication vial.

FIG. 17 illustrates a vial adapter, such as the vial adapter shown in FIGS. 12-14, containing a medication vial.

FIG. 18 illustrates the vial adapter of FIG. 17 with the dust cap removed.

FIGS. 19a-c illustrate a needle-free injector assemblies.

FIG. 20 illustrates an injector assembly used with a wand-type needle-free injector.

FIG. 21 illustrates a vial adapter in an adapter assembly, with a vial inserted.

FIG. 22 illustrates an exploded view of one embodiment of the vial adapter assembly.

FIG. 23 illustrates an exploded view of another embodiment of the vial adapter assembly.

### DETAILED DESCRIPTION

Provided herein are a medicine vial adapter and assembly for a needle-free injector. The vial adapter may be used with known needle-free injectors, such as in Menassa, supra. In this regard, a needle-free injector such as a “gun-type” or an injector such as a “hose-type” may be used in certain embodiments. However, any suitable needle-free injector which can be fitted with a vial adapter to form an assembly as described herein may be contemplated.

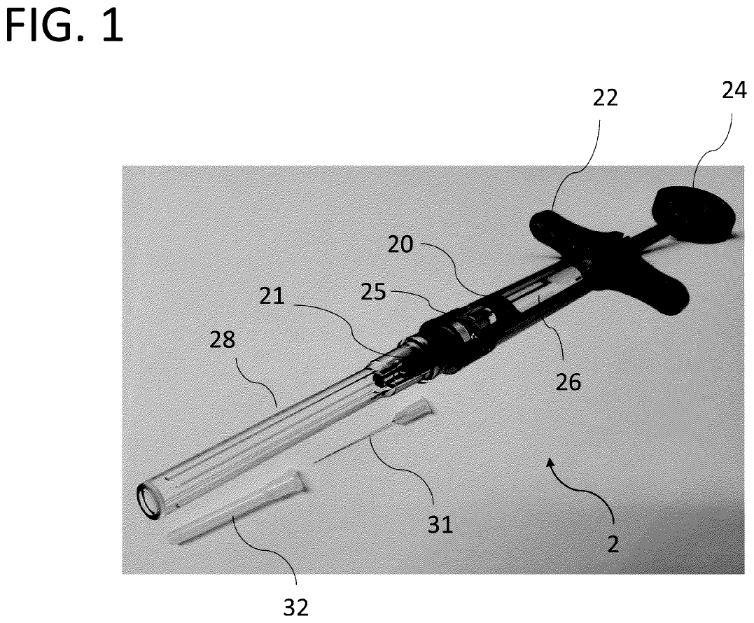

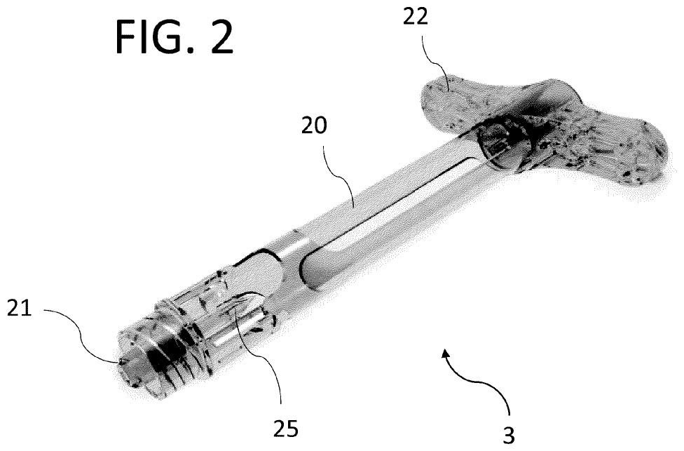

FIGS. 1, 2 and 5 illustrate various tools and adapters, including those which are typically or may be used for administering a medicament in dental applications. FIG. 1 illustrates a typical dental syringe and needle. Traditionally, oral anesthetic and the like is administered by a syringe 2 comprising a plunger head 24 and hand grip 22. Vial holder 20 holds vial 26, which is typically a glass cartridge or ampoule. After a vial is inserted into the vial holder, one or more spikes 25 rupture a seal on the vial, allowing for passage of the medicament within the vial through the luer lock 21 and into a needle 31 (with protective needle cover 32, directed by a nozzle 28. The medicament from the vial is administered when a dentist pushes on the plunger head, releasing the drug through the needle injected into the patient's tissues. FIG. 2 shows the syringe illustrated in FIG. 1 without the plunger head, needle or nozzle.

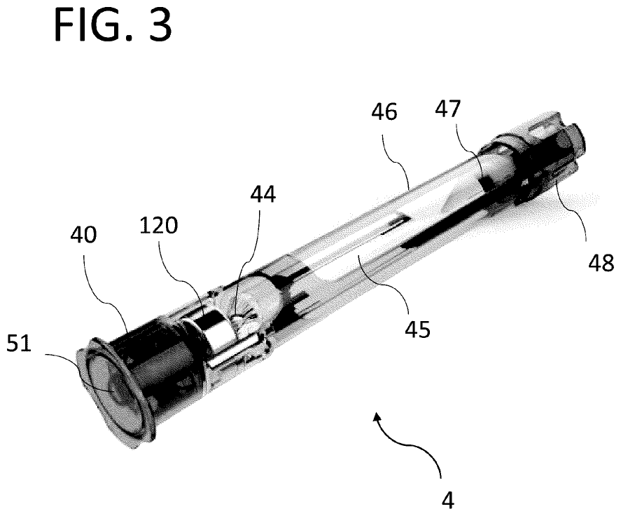

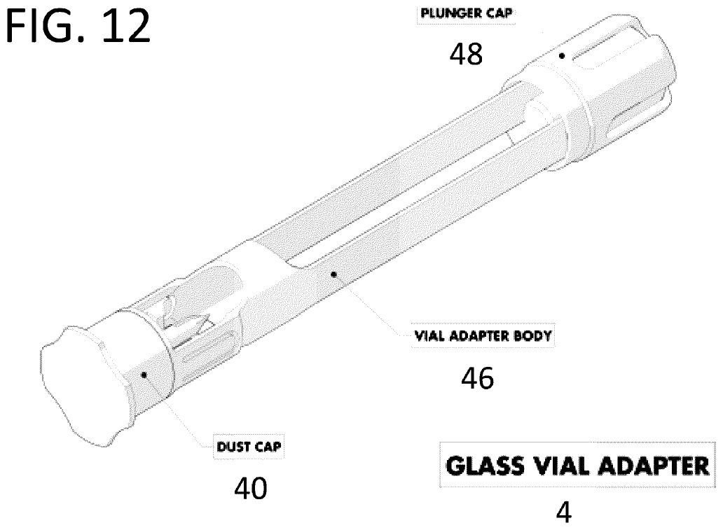

FIGS. 3 and 12 illustrate one embodiment of a vial adapter as provided herein. Vial adapter 4 generally comprises a vial adapter body 46. In some embodiments, the vial adapter body 46 is made of plastic or other durable material. Ideally, the vial may be at least partially transparent to permit a user to see inside the adapter (including the vial and the contents thereof); in some embodiments, a transparent window 45 may be provided.

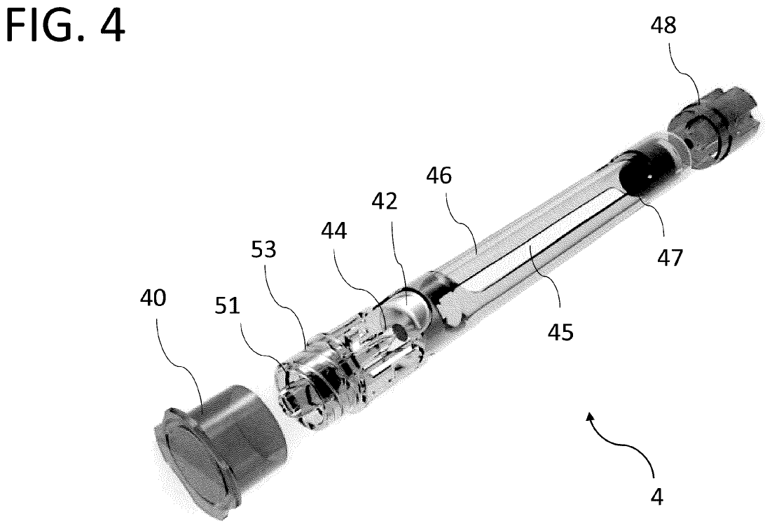

FIG. 4 shows an exploded view of the vial adapter of FIG. 3. Dust cap 40 may be threaded to attach to the vial adapter body via threads 53.

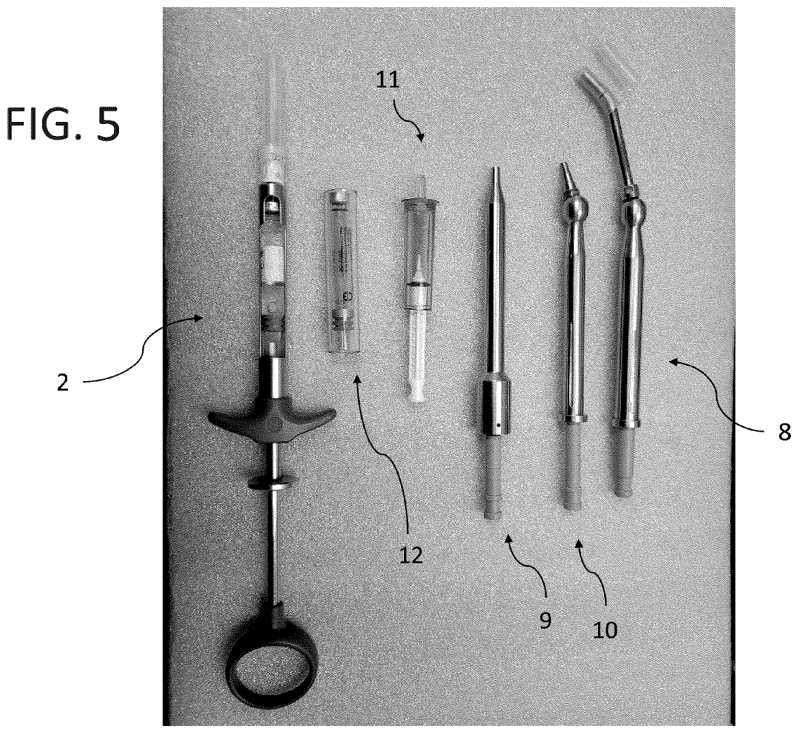

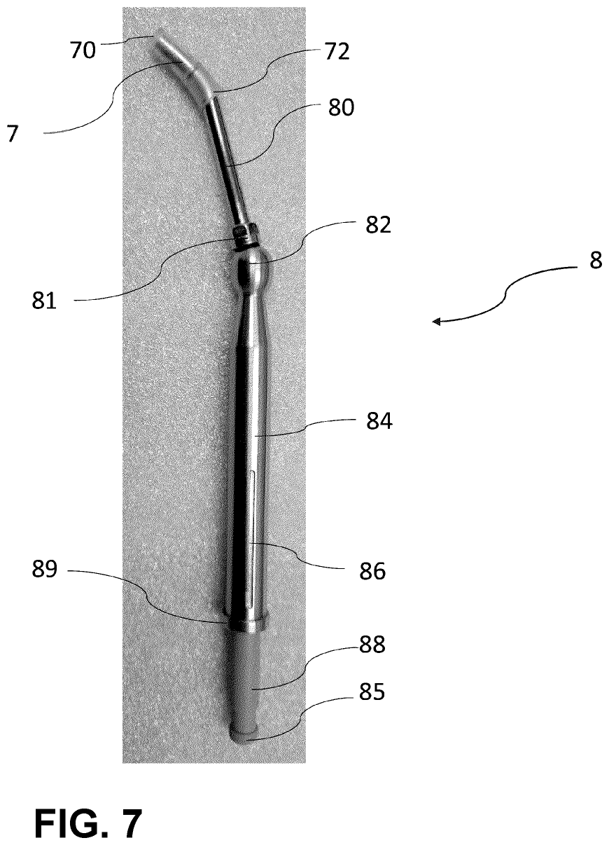

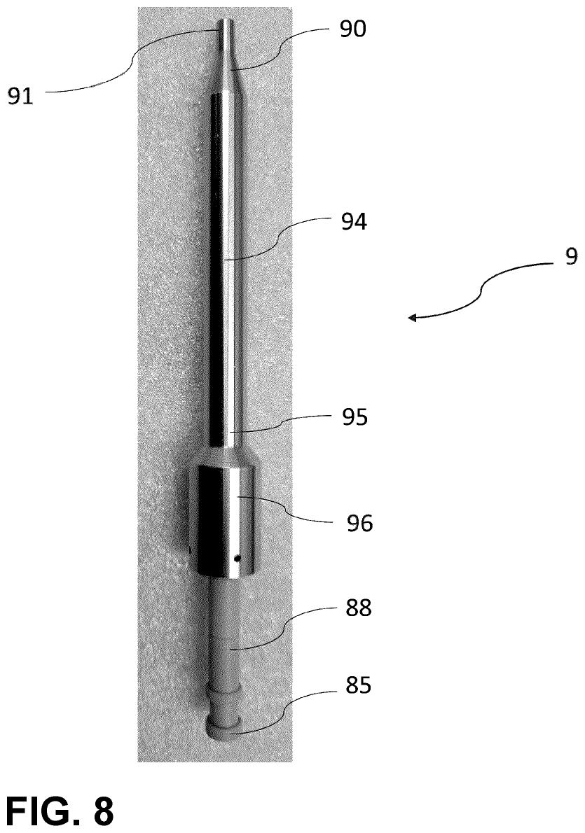

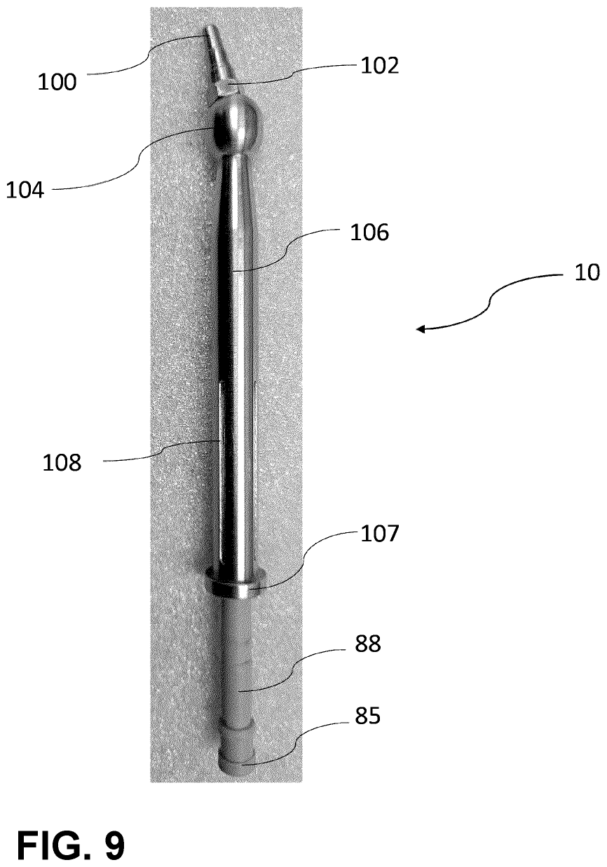

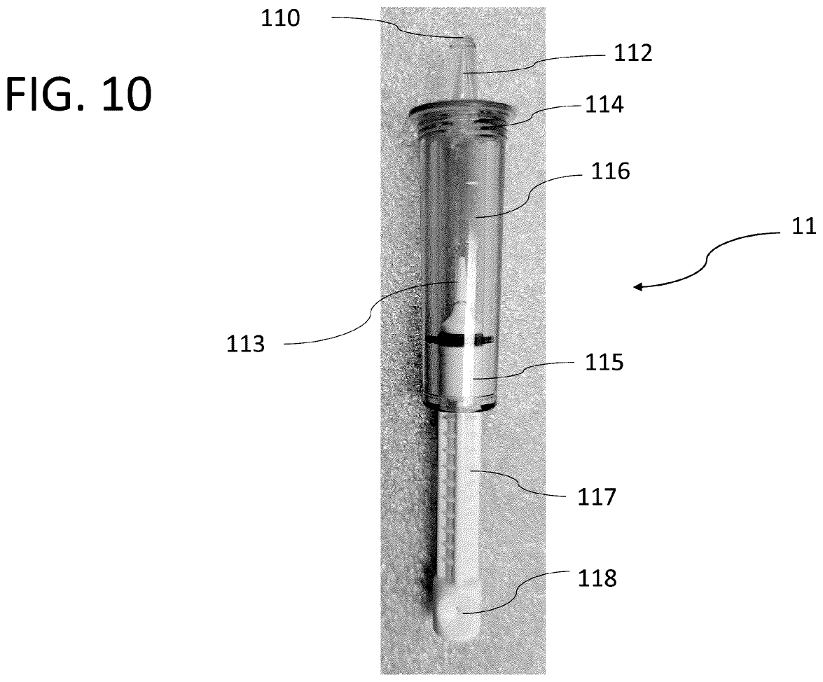

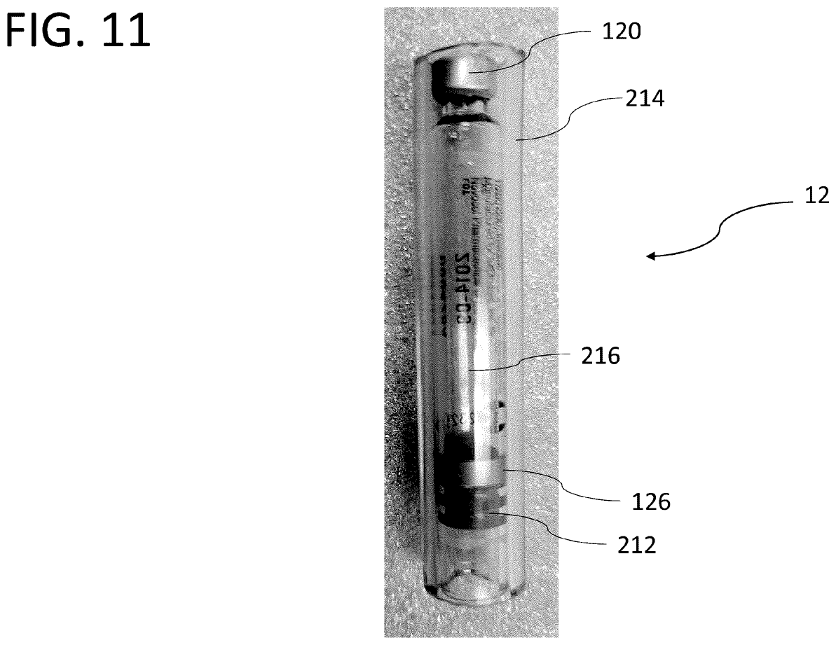

FIG. 5 shows a panel of dental tools, including a typical dental syringe 2 (FIG. 1) and various vial (cartridge) adapters and tools which may be used with a needle-free injector, including dental needle-free stainless steel cartridges and pistons having a straight nozzle 9 (FIG. 8), angled tip nozzle 10 (FIG. 9) and long angled nozzle 8 (FIG. 7). Vial assembly 12 in protecting tube (FIG. 11) and vial cartridge 11 (FIG. 10) are also shown. The vial cartridge 11 shown in FIG. 10 may be used with liquid medicament added directly thereto. In one embodiment, the vial cartridge 11 is disposable. Ideally, these tools are used for dental applications (e.g. injecting anesthetic into oral tissue), but may be used for other health-related injection applications as appropriate.

The tools shown in FIGS. 7-9 are attachments typically used with a needle-free injector, whereby they are used for aspirating/siphoning medicament via the tip of the nozzle end, from a carpule or other vial exterior to the injector. The medicament is then injected one or more times from the needle-free injector using these tools. In some embodiments, a carpule may be connected to a vial adapter which is then connectable to one of the stainless steel cartridges as shown therein for retrieving the medicament from the vial to be inserted into the cartridge for injection.

These dental tool adapters (8, 9 and 10) generally comprise a barrel (84, 94, 106). In use, medicament is drawn therein as described above, for injection. The barrel is releasably connected (such as by a threaded connection or the like, e.g. 89, 96, 107) to a needle-free injector power unit. A piston 88 impacts onto the interior of the barrel upon force impacted onto piston end 85. The nozzles at the injection end may vary: angled nozzle 80 connected by nut 81 via connector 82 (see FIG. 7); straight barrel with small nozzle 90 and nozzle tip 91 (see FIG. 8); small angled nozzle 100 connected to the barrel 106 at connector 104 and nut 102 (see FIG. 9). Air is purged out of the tool by priming the injector as required.

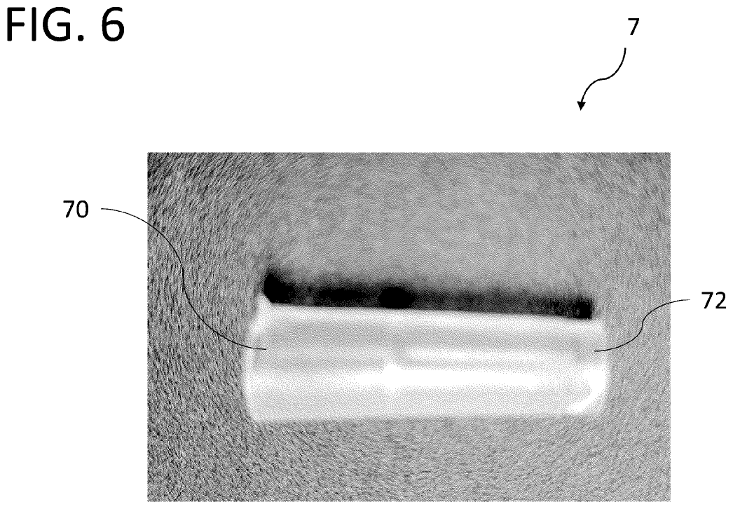

FIG. 6 shows a soft tip 7 having an open end 70 (which covers the tip of the nozzle being used in the patient) and a tip end 72.

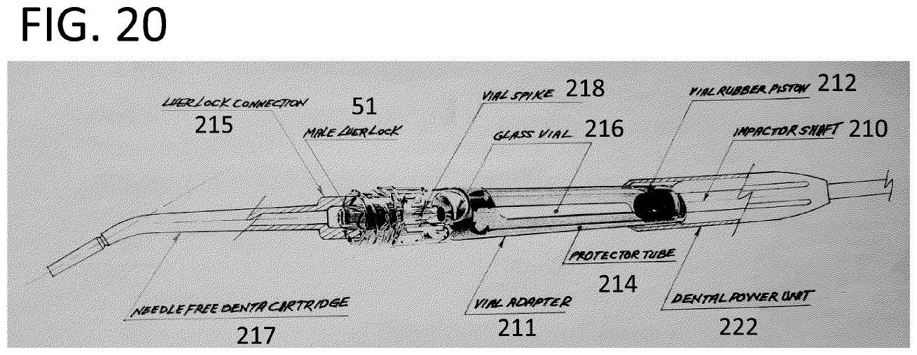

FIG. 11 shows one embodiment of a vial cartridge assembly 12 as typically used and which is inserted into the vial adapter. The vial cartridge assembly 12 comprises a vial 216 and vial protector tube 214, and a sealed cap 120 from which the medicament is expelled through. Plunger 212 is impacted by a piston (see FIGS. 20, 22, 23) from the needle-free injector power unit when in use, causing plunger end 126 to force medicament out of the vial. The vial 125 is usually glass, but can be other suitable material. The protector tube 214 is typically made of glass, plastic or other suitable material.

FIG. 12 shows a general view of one embodiment of a glass vial adapter having a vial adapter body 4 such as shown in FIG. 4, but without a dust cap or vial. At one (plunger) end of the adapter there may be a plunger cap 48 with plunger 47. At an opposite (injection) end there is typically a male luer lock 51 for engaging with a disposable nozzle or other adapter from which the medicament leaves the vial to be injected into the subject. One or more bottle spikes 44 at this opposite end pierce the covering (e.g. rubber stopper or other member which sealingly engages the vial to be used) of the vial when inserted in to the vial adapter. Locking tab 41 may be provided. A dust cap 40 may be used to minimize dust and other contaminants from the tip of the vial adapter body at the injection end.

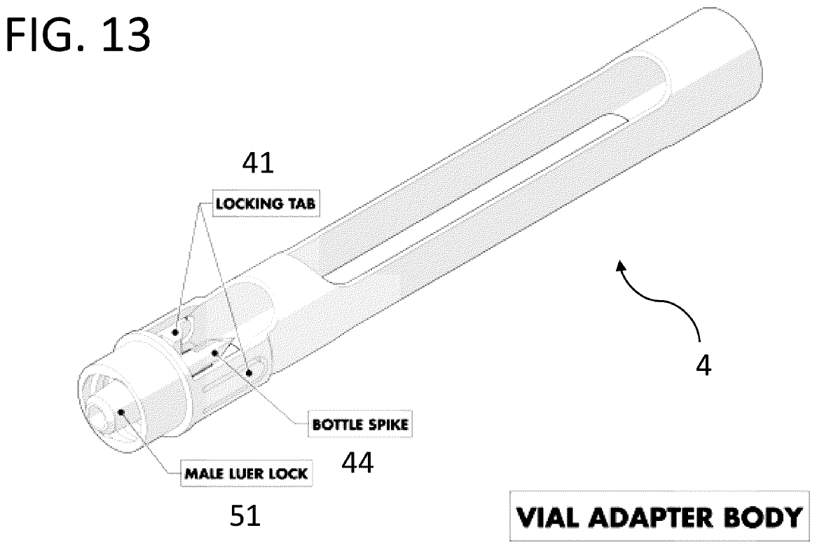

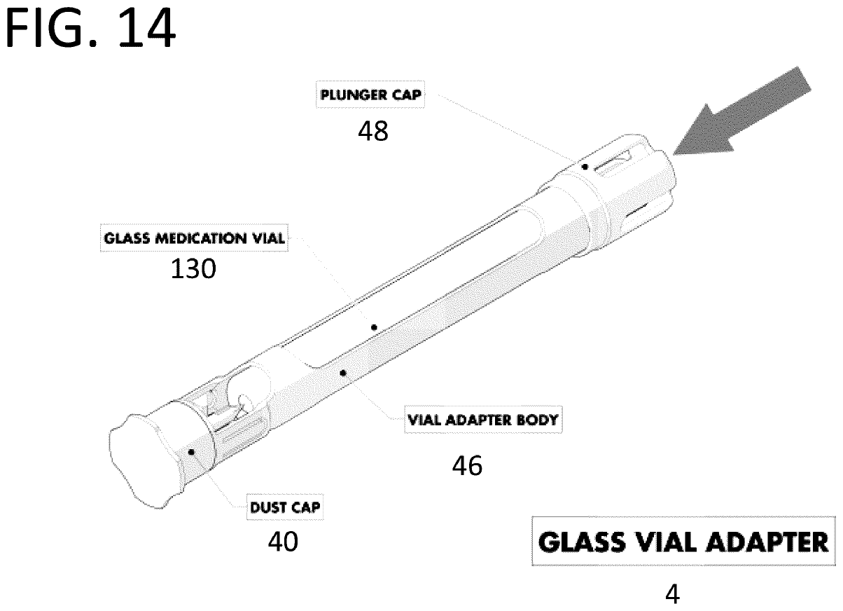

FIGS. 13 and 14 shows one embodiment of the vial adapter body 4 with plunger cap 48 and vial (glass medication vial 130 shown) inserted into the vial adapter body 46 with a dust cap 40 attached. The arrow indicates the direction of the plunger force from a needle-free injector onto the glass vial within the adapter. Once the plunger cap 48 is attached, a user presses (firmly) on the plunger cap to drive the bottle spike 44 into the tip of the vial to pierce an opening in the rubber vial seal. This will expel a precise amount of liquid to purge the vial adapter for use.

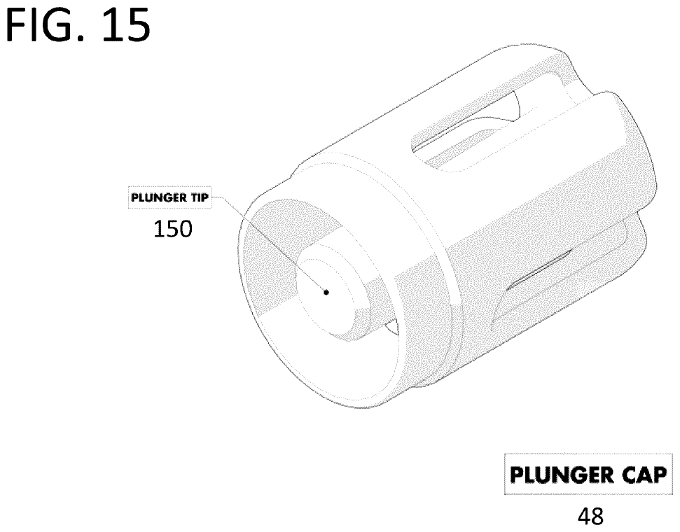

FIG. 15 shows a typical plunger cap in isolation. The plunger cap 48 is generally cylindrical and may comprise a coaxial boss in a centre region. The plunger cap is suitably adaptable for attaching to a needle-free injector mechanism. The coaxial plunger tip is used to press on the rubber stopper in the vial to both drive the vial forward onto the spike and after it has pierced to expel a calculated volume of liquid to clear the air from the vial adapter body.

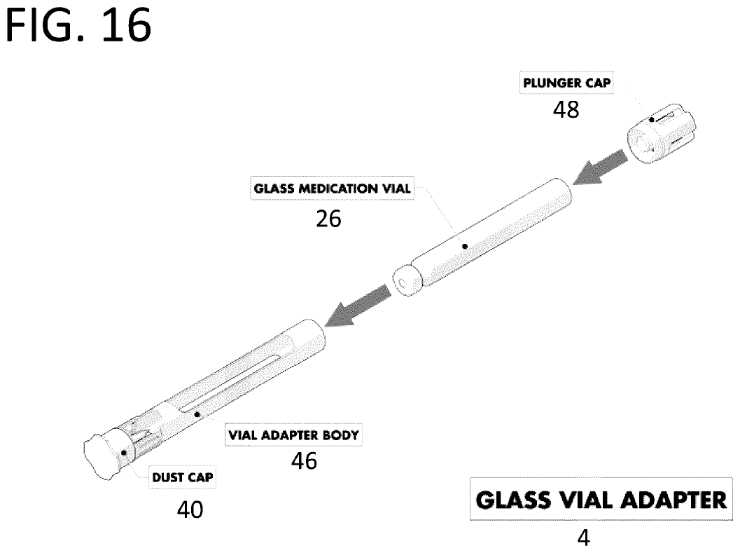

FIG. 16 shows an exploded view of one embodiment of a glass vial adapter as described herein when used with a glass medication vial. The arrows show the directions for inserting the glass medication vial into an opening at the back end of the vial adapter body distal to the port of injection (i.e., closer to the needle-free injector), and the direction for addition of the plunger cap (such as threaded) on to that back end once the glass medication vial has been inserted into the adapter.

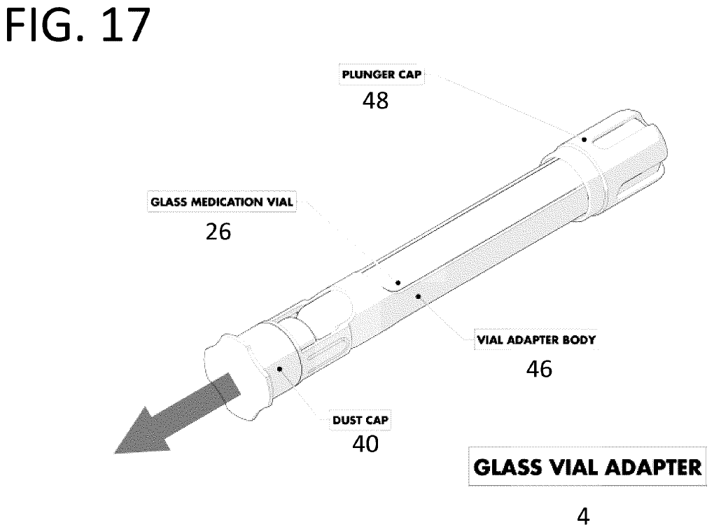

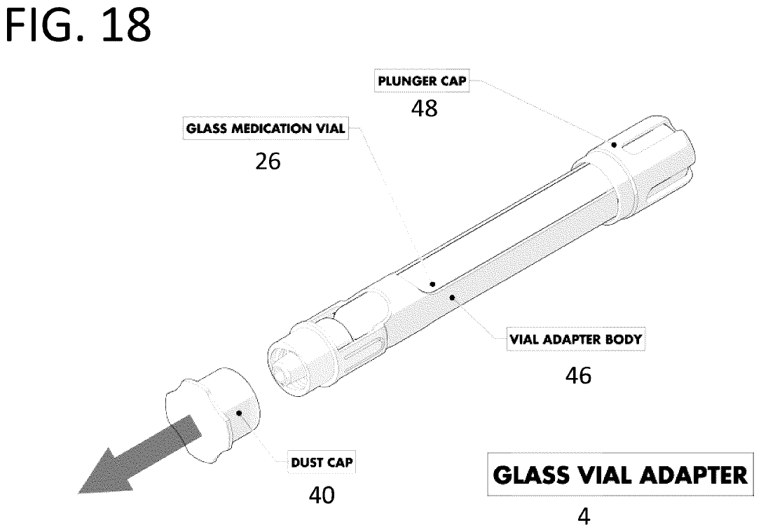

FIG. 17 shows one embodiment of a glass vial adapter with a glass medication vial inserted therein. The arrow indicates the direction of expulsion of the medicament once the dust cap is removed. FIG. 18 shows the vial adapter with the glass vial inserted and the dust cap removed (in the direction of the arrow). Once the vial adapter is properly purged (to remove air and dust), the medication may be drawn in automatically for injection.

To gain a better understanding, examples of needle-free injector assemblies as described herein are set forth below. These examples are for illustrative purposes only, and they should not limit the scope of the present application in any way.

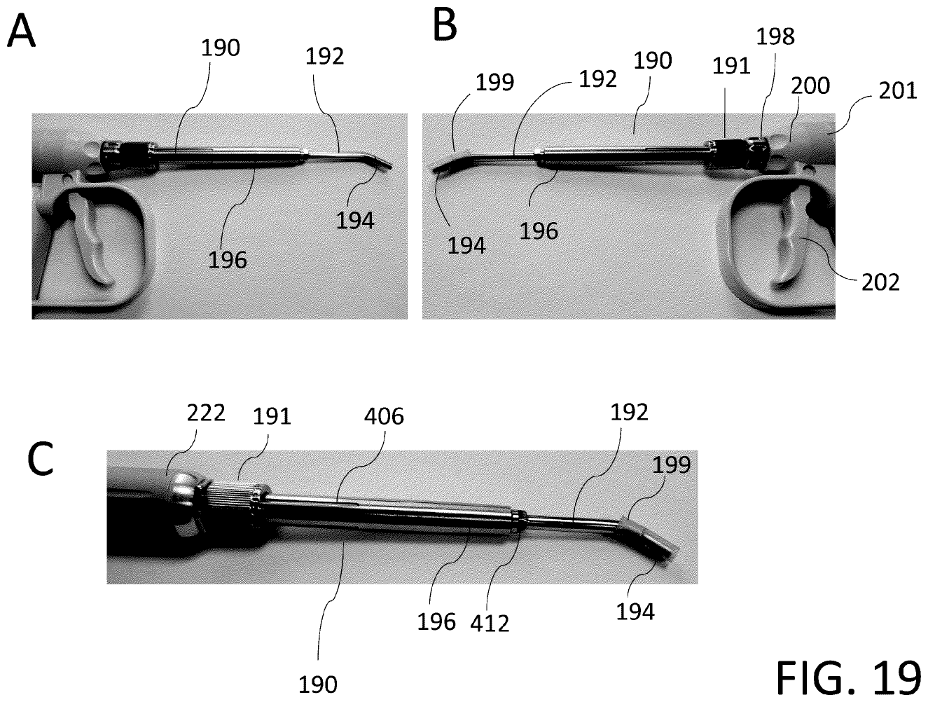

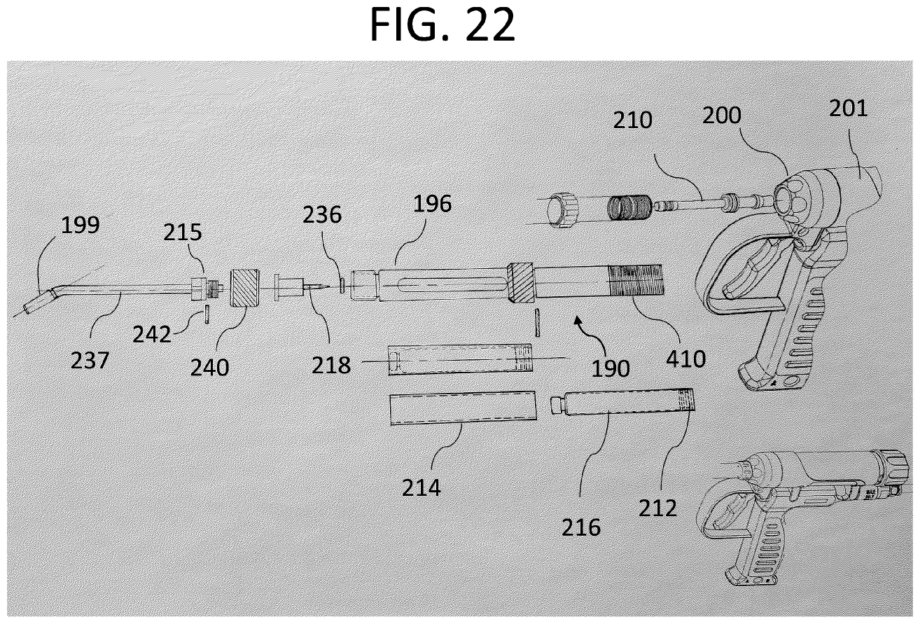

FIGS. 19 and 22 illustrate certain embodiments of a needle-free injector assembly comprising a single barrel and injection nozzle, such as a dental injection nozzle for example. FIG. 19B shows a side view of one embodiment of the assembly with a protective nozzle soft tip 199, and FIG. 19A shows a side view of the assembly without the nozzle cap. A “gun-type” needle-free injector 201 comprises a trigger 202 which is used to “fire” the injector piston (not shown). FIG. 19C shows one embodiment of a “wand-type” needle-free injector 222, such as described in Menassa, supra. In the embodiment shown, the injection barrel comprises a single machined barrel 190. In this regard, the barrel 190 comprises a front vial adapter component 196. A threaded nut 198 on a rear end of barrel may be used to engage with a chuck 200 on the injection end of the needle-free injector power unit 201 and locks via a threaded end 410, thereby locking the special degree nozzle adapter nut 191 with the injector unit at a proximal end (closer to the injector itself), such that the impactor shaft 210 (shown in FIG. 22) is able to contact with the vial at rubber piston 219. A vial adapter as described herein (such as shown in FIGS. 12-14 and 16-18), comprising a vial of medicament to be administered (not shown), may be inserted into an opening, such as at the front end 196 of the barrel 190 (which may or may not already be connected to the power unit 201), i.e., at the end closest to the patient injection end of the unit. In some embodiments, the barrel may comprise a window or similar opening 406 which permits a user to see when the vial is inserted and how much medicament remains in the vial. A nozzle 192 extends from the distal (injection) end of the nozzle 196 via nozzle adaptor 412. A nozzle soft tip 199 may be provided which covers the nozzle head 194 to more comfortably contact the patient's tissue area. The nozzle and accessories therefor are ideally disposable, such as made of plastic or the like, and are typically discarded after use to prevent contamination between patients. As the nozzle is locked and twisted onto the vial adapter (such as via a nut 240), a vial spike 218 punctures a rubber seal at that distal end of the vial to release medicament from the vial.

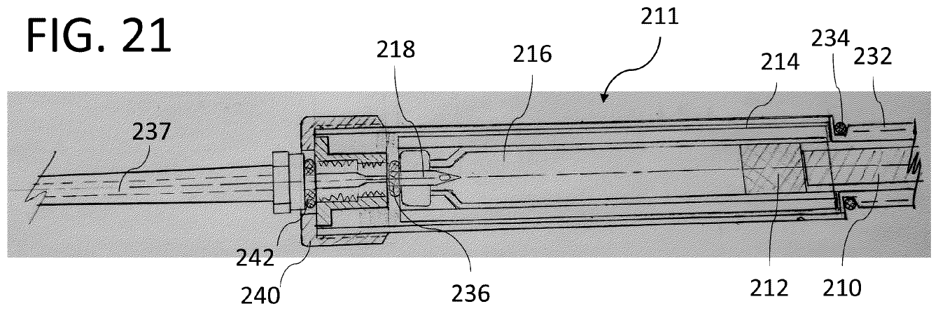

FIGS. 20 and 21 show another embodiment of an injector vial assembly (FIG. 20) and close up of the vial adapter (FIG. 21) inserted into the injection barrel of the injector assembly. In this example, the injector barrel is comprised of two separate components attached to each other: a vial adapter 211 and an impactor barrel component of the dental power unit 222 (or other suitable injection power unit of the like). As shown, a “wand-” or “hose-type” needle-free injector power unit is shown, but other power units may be contemplated. A vial adapter 211 (such as, for example, shown in FIGS. 12-14 and 16-18) is attachable to dental power unit 222. In one embodiment, and unlike the injection assembly shown in FIGS. 19 and 22, a glass vial or carpule 216 of medicament may be inserted into the protector tube 214 component of the vial adapter from the back end (i.e. at the end closest to the power unit, opposite the distal injection end nearest a patient). A nozzle 217 (also referred to herein as a “needle free dental cartridge”, see e.g. in FIG. 20) is attachable, typically screwed, onto a luer lock 51 at the distal, front end of the vial adapter. FIG. 21 shows a vial adapter (such as, in one embedment, a dental vial adapter) for use with gun-type needle-free injector. In one embodiment, the vial adapter 211 is then threaded onto the power unit of the needle-free injector 232, using an O-ring 234 or similar gasket to seal the connection. O-rings 236, 242 may also be used to seal the vial at the expelling end. The needle-free disposable nozzle, such as a plastic nozzle 237, is connected to the vial adapter assembly (at the vial adapter end) via a nut 240 and can ideally be hand-tightened; once it is tightened, the vial spike 218 punctures the seal of the glass vial 216, releasing medicament therefrom. In one embodiment, the nozzle 237 is a dental nozzle.

Prior to injection the vial adapter and injection assembly may be purged of air before use. Two exemplary methods of assembly and removing the air before injecting are as follows for two of the embodiments of the injector assembly described herein:

Example Method 1: INJECTOR ASSEMBLY (e.g. Single Machined Injector Barrel/Vial Adapter; See FIGS. 19, 22):
Step 1—Install a glass vial containing the medicament and tube protector inside the
   vial adapter (front i.e. nozzle end).
Step 2—Connect a disposable cartridge (nozzle) into the vial adapter (spike end).
Step 3—Install a nut to secure disposable nozzle inside the vial adapter at the same time; the spike will perforate the rubber stopper of the glass vial and
   some medicament (e.g. anesthesia liquid) will fill the disposable nozzle.
Step 4—Turn the knob of the power unit (not shown) clockwise to push the rubber
   piston of the glass vial with the impactor shaft. This will fill the disposable nozzle and at the same time release the air from the front of the disposable nozzle.
Step 5—Turn the knob of the power unit (not shown) counterclockwise to adjust the impact space needed for proper injection site.
   Example Method 2: INJECTOR ASSEMBLY (e.g. Two Component Vial Adapter/Injector Assembly, See FIGS. 20, 21, 23)
Step 1—Connect a nozzle onto the luer lock of the vial adaptor.
Step 2—Install a glass (e,g, dental) carpule and protector tube inside the vial adapter from the back.
Step 3—Use the plunger cap to secure the glass dental carpule and protector tube
   inside the vial adapter, at the same time; the spike will perforate the rubber stopper of the glass dental carpule and some medicament (e.g. anesthesia liquid) will fill the disposable cartridge (nozzle)—Note: a very small amount of the medicament will be lost but this is part of the expulsion process which also expels excessive air with it from the injector assembly.
Step 4—Remove the plunger cap on the back of the vial adapter and connect the vial adaptor to the injector power unit by turning clockwise, the impactor shaft will push the rubber piston of the glass dental carpule, to fill the nozzle and at the same time release the air from the front of the nozzle.
Step 5—Turn counter clockwise the vial adaptor one or two turns to adjust the impact space needed for the proper injection impact.

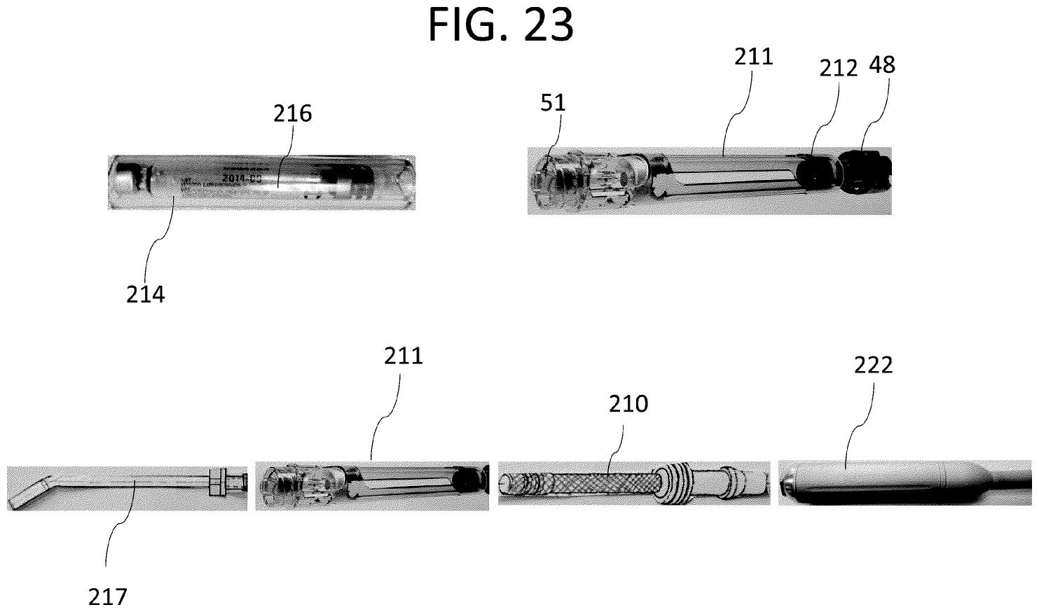

FIG. 23 is an exploded view of the injector assembly of FIG. 20. In one embodiment, the nozzle 217 is attached to the luer lock 51 of the vial adapter 211, thereby connecting the nozzle with the vial adapter. The carpule 216 (such as in glass protector tube 214) can be inserted into the vial adapter from the rear end. The plunger cap 48 is then pushed to secure the carpule 216/glass and protector tube 214 inside the vial adapter 211, thereby pushing the carpule into the spike. Some fluid medicament is typically pushed into the nozzle, getting the injector assembly ready. The plunger cap is then removed from the back of the vial adapter, and then the vial adapter is connected to the components of the power unit (210, 222), typically by turning the vial adapter clockwise to secure. The impactor shaft 210 abuts the rubber plunger 212 at the back end of the vial adapter. for purging the system and priming it for injection.

### Example Embodiments

Embodiment 1: An adapter for a needle-free injector comprising: a vial cartridge holder comprising a protector tube for holding a vial of medicament to be injected; a cutting means, such as a spike, knife or other sharp, for perforating a seal on the vial to form a perforation which allows the medicament to be injected therethrough; a luer lock at one end of the adapter for releasably connecting with a nozzle.

Embodiment 2: The adapter of embodiment 1 as a dental tool for injecting an anesthetic or other medicament to a tissue in an oral cavity of a patient in need thereof.

Embodiment 3: The adapter of embodiment 1, wherein the needle-free injector is a gun-type or wand-type needle-free injector.

Embodiment 4: The adapter of any one of embodiments 1-3, wherein the nozzle is straight or angled, typically disposable, and is used for dispensing medicament from the vial to the tissue of patient, such as oral tissue.

Embodiment 5: A needle-free injector assembly comprising: a barrel attached at a first end to a power injector unit having an impactor shaft therein; a vial adapter releasably connected at or insertable into a second of the barrel for housing a vial (such as a glass vial) of medicament to be injected; and a nozzle for releasably attaching to an injection end of the vial adapter.

Embodiment 6: The needle-free injector assembly of embodiment 5, wherein the vial is inserted into vial adapter at a rear end prior to attachment to the second end of the barrel.

Embodiment 7: The needle-free injector assembly of any one of embodiments 5 to 6, for use as a dental tool in dental applications.

Embodiment 8: A needle-free injector assembly comprising: a barrel, such as a single machined barrel, releasably connected to a power injection unit, the barrel comprising a shaft for housing an impactor shaft from a power injection unit at the rear end, and a vial adapter component at an injection end of the barrel for housing a vial adapter comprising a vial of medicament; and a nozzle which is releasably attachable to the injection end of the barrel, whereby by attaching the nozzle to the injection end, a spike perforates a seal on the vial thereby releasing medicament.

Embodiment 9: The needle-free injection assembly of embodiment 7, wherein the vial is inserted into the injection end of the vial after the barrel is connected to the power unit.

Embodiment 10: The needle-free injector assembly of any one of embodiments 9 to 10, for use as a dental tool in dental applications.

Embodiment 11: A dental tool releasably attachable to a needle-free injector power unit comprising a barrel for housing a piston/rod assembly which is impacted by the power unit, and for housing medicament which is aspirated/siphoned therein, for injection through a nozzle end of the tool.

The above disclosure and figures are intended to be illustrative and not exhaustive. The description will suggest many variations and alternatives to one of ordinary skill in the art. Those familiar with the art may recognize other equivalents to the specific embodiments described herein within, without departing from the spirit and scope thereof.

1. An adapter for a needle-free injector comprising:
a vial cartridge holder comprising a protector tube for holding a vial of medicament to be injected;
a cutting means for perforating a seal on the vial to form a perforation which allows the medicament to be injected therethrough; and
a lock at one end of the adapter for releasably connecting with a nozzle.

2. The adapter of claim 1, wherein the cutting means is a spike, knife or other sharp.

3. The adapter of claim 1, wherein the lock is a luer lock.

4. The adapter of claim 1, for use as a dental tool for injecting the medicament, such as an anesthetic, to a tissue in an oral cavity of a patient in need thereof.

5. The adapter of claim 1, wherein the needle-free injector is a gun-type or wand-type needle-free injector.

6. The adapter of claim 1, wherein the nozzle is straight or angled, and is used for dispensing medicament from the vial to the tissue of patient, such as oral tissue.

7. The adapter of claim 6, wherein the nozzle is disposable.

8. A needle-free injector assembly comprising:
a barrel attached at a first end to a power injector unit having an impactor shaft therein;
a vial adapter releasably connected at or insertable into a second of the barrel for housing a vial of medicament to be injected; and
a nozzle for releasably attaching to an injection end of the vial adapter.

9. The needle-free injector assembly of claim 8, wherein the vial is a glass vial.

10. The needle-free injector assembly of claim 8, wherein the vial is inserted into vial adapter at a rear end prior to attachment to the second end of the barrel.

11. The needle-free injector assembly of claim 8, for use as a dental tool in a dental application.

12. A needle-free injector assembly comprising:
a barrel releasably connected to a power injection unit, the barrel comprising a shaft for housing an impactor shaft from a power injection unit at the rear end, and a vial adapter component at an injection end of the barrel for housing a vial adapter comprising a vial of medicament; and
a nozzle which is releasably attachable to the injection end of the barrel, whereby by attaching the nozzle to the injection end, a spike perforates a seal on the vial thereby releasing medicament.

13. The needle-free injector assembly of claim 12, wherein the barrel is a single machined barrel.

14. The needle-free injection assembly of claim 12, wherein the vial is inserted into the injection end of the vial after the barrel is connected to the power unit.

15. The needle-free injection assembly of claim 12, for use as a dental tool in dental applications.

16. (canceled)

17. The adapter of claim 2, wherein the lock is a luer lock.

18. The adapter of claim 17, wherein the needle-free injector is a gun-type or wand-type needle-free injector.

19. The adapter of claim 18, wherein the nozzle is straight or angled, and is used for dispensing medicament from the vial to the tissue of patient, such as oral tissue.
